(19) United States
(12) Patent Application Publication
(10) Pub. No.: US 2021/0046018 A1
(43) Pub. Date: Feb. 18, 2021

(54) RESVERATROL MEDICATION FOR THE TREATMENT OF OCULAR PAIN AND METHOD OF USE THEREOF

(71) Applicant: TED'S BRAIN SCIENCE, INC., Dallas, TX (US)

(72) Inventors: SUE A. AICHER, PORTLAND, OR (US); GREGORY O. DUSSOR, AUSTIN, TX (US); THEODORE J. PRICE, AUSTIN, TX (US); DENNIS I. ROBBINS, DALLAS, TX (US); DAVID H. HITT, II, DALLAS, TX (US); DAVID H. HITT, III, DALLAS, TX (US)

(73) Assignees: TED'S BRAIN SCIENCE, INC., DALLAS, TX (US); BOARD OF REGENTS, THE UNIVERSITY OF TEXAS SYSTEM, AUSTIN, TX (US); OREGON HEALTH AND SCIENCE UNIVERSITY, PORTLAND, OR (US)

(21) Appl. No.: 16/992,988

(22) Filed: Aug. 13, 2020

Related U.S. Application Data
(60) Provisional application No. 62/888,151, filed on Aug. 16, 2019.
(60) Provisional application No. 62/893,072, filed on Aug. 28, 2019.

(51) Int. Cl. A61K 31/05; A61K 9/00; A61K 47/32; A61K 47/38; A61K 47/36; A61K 31/196; A61K 31/407; A61K 45/06; A61K 31/56; A61K 31/573; A61K 31/569

(52) U.S. Cl. CPC A61K 31/05; A61K 9/0048; A61K 47/32; A61K 47/38; A61K 47/36; A61K 31/569; A61K 31/407; A61K 45/06; A61K 31/56; A61K 31/573; A61K 31/196

(57) ABSTRACT

The present disclosure provides ophthalmic compositions containing resveratrol. Also provided are methods of using ophthalmic resveratrol compositions to treat acute ocular pain, chronic ocular pain, dry eye disease, and/or photophobia.

### CROSS-REFERENCE TO RELATED APPLICATIONS

This application claims the benefit of priority to U.S. Provisional Application Ser. No. 62/888,151, entitled “RESVERATROL MEDICATION FOR THE TREATMENT OF OCULAR PAIN AND METHOD OF USE THEREOF,” filed on Aug. 16, 2019 and U.S.

Provisional Application Ser. No. 62/893,072, entitled “RESVERATROL MEDICATION FOR THE TREATMENT OF OCULAR PAIN AND METHOD OF USE THEREOF,” filed on Aug. 28, 2019, the disclosures of which are incorporated herein by reference in their entireties.

### BACKGROUND

More than 500,000 individuals undergo laser vision correction surgery each year in the United States, and more than 9 million Americans have experienced laser vision correction surgery since the methods were developed in the 1990s. Two distinct methods are used: photorefractive keratectomy (PRK) and laser-assisted in situ keratomileusis (LASIK). These procedures use slightly different approaches to access the inner corneal structures, but both involve severing the nerves in the corneal epithelium. The pain caused by PRK or LASIK is quite intense in the first 24-72 hours post-operatively. Using a numeric rating scale with 0=no pain and 10=worst pain, most patients rate their pain as greater than 6, and many report the pain to be “unbearable,” even after analgesic treatment. Pain severity is not related to gender or anxiety levels before surgery and is not effectively managed by topical opioids, non-steroidal anti-inflammatory compounds, or preoperative treatment with gabapentinoids. The standard of care is usually administration of systemic opioids, which poses a risk for addiction.

In addition to acute and chronic pain, laser vision correction surgery produces photophobia (an aversion to light, resulting from photoallodynia) and dry eye, the latter likely because damage to corneal sensory nerves also disrupts afferent signaling that causes tear production in response to the sensation of dryness at the ocular surface. The sensory and autonomic mechanisms involved in protecting the ocular surface are referred to as the lacrimal functional unit, and the sensory nerves involved seem to be identical to the nerves that relay ocular pain.

It is apparent that ocular surgical procedures would benefit from better analgesic medications, and that effective ocular pain management represents a large, unmet clinical need. The present disclosure addresses this need.

### BRIEF SUMMARY OF THE INVENTION

In one embodiment, an ophthalmic composition is provided. The ophthalmic composition includes about 0.001% to about 5% (w/v) resveratrol, about 0.001% to about 5% (w/v) of at least one pharmaceutically acceptable excipient, and an aqueous vehicle.

In another embodiment, a method of treating a disease or disorder of the eye is provided. The disease or disorder of the eye includes acute ocular pain, chronic ocular pain, dry eye disease, or photophobia. The method includes administering a therapeutically effective amount of a composition that includes about 0.001% to about 5% (w/v) resveratrol, about 0.001% to about 5% (w/v) of at least one pharmaceutically acceptable excipient, and an aqueous vehicle to an eye of a subject in need thereof.

### BRIEF DESCRIPTION OF THE FIGURES

The drawings illustrate generally, by way of example, but not by way of limitation, various embodiments of the present application.

FIG. 1 is a diagram of corneal sensory nerves and reflex pathways.

FIG. 2 is a diagram of adenosine monophosphate-activated protein kinase (AMPK) signaling.

FIG. 3 is a diagram of a corneal abrasion method performed on an anesthetized rat.

FIG. 4 is a graph representing acute hyperalgesia after corneal abrasion injury.

FIGS. 5A-5C are diagrams of hematoxylin and eosin (H&E) stains showing loss of epithelial cells 24 hours after abrasion injury (FIG. 5B) compared to control (FIG. 5A), illustrating that capsaicin application (FIG. 5C) does not alter overall epithelial structure.

FIGS. 6A and 6B are graphs showing efficacy of resveratrol medication for the treatment of ocular pain, chronic ocular pain, dry eye disease, and/or photophobia.

### DETAILED DESCRIPTION OF THE INVENTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

As used herein, the term “composition” or “pharmaceutical composition” refers to a mixture of at least one compound described herein with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

As used herein, the term “efficacy” refers to the maximal effect (Emax) achieved within an assay.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985, Easton, Pa.), which is incorporated herein by reference.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human. In other embodiments, the patient, subject or individual is a non-human animal.

As used herein, the term “potency” refers to the dose needed to produce half the maximal response (ED₅₀).

A “therapeutic” treatment is a treatment administered to a subject who exhibits signs of pathology, for the purpose of diminishing or eliminating those signs.

As used herein, the term “treatment” or “treating” is defined as the application or administration of a therapeutic agent, i.e., a compound or compounds as described herein (alone or in combination with another pharmaceutical agent), to a patient, or application or administration of a therapeutic agent to an isolated tissue or cell line from a patient (e.g., for diagnosis or ex vivo applications), who has a condition contemplated herein or a symptom of a condition contemplated herein, with the purpose to cure, heal, alleviate, relieve, alter, remedy, ameliorate, improve or affect a condition contemplated herein, or the symptoms of a condition contemplated herein. Such treatments may be specifically tailored or modified, based on knowledge obtained from the field of pharmacogenomics.

### Topical Compositions

Resveratrol is a natural polyphenol that is found in red grapes, wine, and other plant sources, and has been shown to attenuate both acute and chronic incision pain as well as prevent the transition to a chronic pain state. Resveratrol is thought to reduce pain by engaging AMP-activated protein kinase (AMPK) and subsequently inhibiting both protein translation and the activity of other kinases. This represents a novel mechanism for pain modulation and has been shown to be effective in several distinct pain models.

Resveratrol is one of the most potent known AMPK activators. It possesses favorable chemical properties for topical dosing and tissue permeation (i.e. it is highly lipophilic), is already viewed as safe for human consumption by the FDA and is approved as an active antioxidant ingredient for topical formulas (non-corneal). Further, it has been shown that topical resveratrol attenuates both acute hyperalgesia following hindpaw incision and also prevents the full development of hyperalgesic priming 14-days after incision, without impairing wound healing. Thus, as described herein, a corneal resveratrol-based, AMPK activator therapeutic represents a novel tool for the treatment of corneal or ocular pain and the prevention of chronic eye pain, photophobia and dry eye. The unique positioning of AMPK as a negative regulator of multiple signaling pathways that are linked to pain make this kinase an ideal target to harness an endogenous regulatory mechanism that may dampen nociceptor excitability and sensitization.

The compositions described herein are, in some embodiments, topical ophthalmic compositions suitable for application to the eye. The topical compositions, in some embodiments, include resveratrol and at least one pharmaceutically acceptable excipient. In one embodiment, the composition contains from about 0.0001% to about 5% (w/v) resveratrol. The resveratrol in the present compositions can be a mixture of cis-resveratrol and trans-resveratrol.

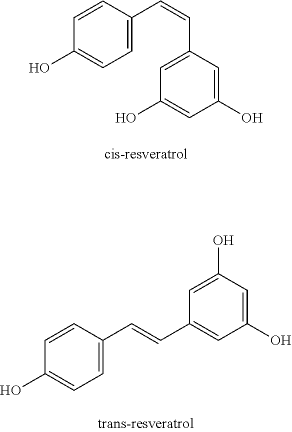 [SMILES: Oc1ccc(/C=C\c2cc(O)cc(O)c2)cc1 ; Oc1ccc(/C=C/c2cc(O)cc(O)c2)cc1]

In certain embodiments, the resveratrol in the composition is at least or equal to about 80%, 85%, 90%, 91%, 92%, 93%, 94%, 95%, 96%, 97%, 98%, 99%, 99.5%, or 99.9% trans-resveratrol, with the balance being cis-resveratrol. The trans-isomer of resveratrol is the more active form of resveratrol. In some embodiments, the resveratrol in the composition is pure trans-resveratrol without any detectable presence of cis-resveratrol.

In some embodiments, the composition contains from about 0.001% to about 5%, about 0.001% to about 4%, 0.001% to about 3%, about 0.001% to about 2%, about 0.001% to about 1%, about 0.001% to about 0.5%, about 0.001% to about 0.25%, about 0.001% to about 0.1%, about 0.001% to about 0.05%, or about 0.001% to about 0.01% (w/v) resveratrol. In some embodiments, the composition contains about 0.0001%, 0.0005%, 0.001%, 0.005%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, or about 5% (w/v) of resveratrol.

The composition may include one or more site 1 sodium channel blockers (S1SCBs), such as tetrodotoxin (TTX) or saxitoxin (STX) individually or in combination with an α2-adrenergic receptor agonists (e.g., dexmedetomidine or clonidine).

The topical composition includes a pharmaceutically acceptable vehicle, such as saline (e.g., 0.9% NaCl in deionized water) or deionized water and can be in the form of eye drops or ointments. The eye drops or ointments, in some embodiments, contain one or more tonicity-adjusting agents in an amount sufficient to cause the final composition to have an ophthalmically acceptable osmolality. Suitable tonicity-adjusting agents include, without limitation, dextrose, glycerin, mannitol, potassium chloride, and sodium chloride. In some embodiments, the topical composition has an osmolality of about 150 to about 450 mOsm, about 200 to about 400 mOsm, or about 250 to about 350 mOsm. The topical compositions, in certain embodiments, have a pH of about 4 to about 8, about 6.5 to about 7.5, or about 6.8 to about 7.2.

In various embodiments, the topical composition has a viscosity of about 0.1 to about 10, about 0.5 to about 5, or about 1 to about 3 cP (centipoise). The topical composition can have a viscosity of about 0.1, 0.5, 1, 2, 3, 4, 5, 6, 7, 8, 9, or 10 cP. The low viscosity of these compositions advantageously ensures that application of the composition to the subject's eye is comfortable, and that little to no blurring of the subject's vision occurs.

As mentioned herein, the topical composition can include one or more pharmaceutically acceptable excipients. Suitable pharmaceutically acceptable excipients include, without limitation, polyvinylpyrrolidone (PVP), polyvinyl alcohol, polyvinyl acrylic acid, hydroxypropylmethyl cellulose, sodium carboxymethyl cellulose, and xanthan gum. Other pharmaceutically acceptable excipients in the topical formulation also include one or more preservatives, chelating agents, buffering agents, surfactants, and/or antioxidants. Suitable preservatives include, without limitation, p-hydroxybenzoic acid ester, benzalkonium chloride, benzododecinium bromide, polyquaternium-1, and the like. Suitable chelating agents include, without limitation, sodium edetate and the like. Suitable buffering agents include, without limitation, phosphates, borates, citrates, acetates, and the like. Suitable surfactants include ionic and nonionic surfactants, such as, without limitation, polysorbates, polyethoxylated castor oil derivatives, and oxyethylated tertiary octylphenol formaldehyde polymer (tyloxapol). Suitable antioxidants include, without limitation, sulfites, ascorbates, BHA, and BHT.

The pharmaceutically acceptable excipients described herein can each be individually present in the composition in an amount of at least, greater than, equal to, or less than about 0.0001%, 0.0005%, 0.001%, 0.005%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 10%, 15%, or 20% (w/v), or any range formed with these amounts. In some embodiments, the total amount of all excipients in the composition is at least, greater than, equal to, or less than about 0.0001%, 0.0005%, 0.001%, 0.005%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 10%, 15%, or 20% (w/v). In some embodiments, the topical composition does not contain one or more of the pharmaceutically acceptable excipients described herein. In certain embodiments, the exclusion of one or more of the pharmaceutically acceptable excipients described herein is beneficial for the stability, efficacy, and/or tolerability (by the subject) of the topical composition. Resveratrol is, in some embodiments, the only therapeutically active agent in the topical composition.

In some embodiments, the composition includes at least one of an analgesic agent, an antipruritic agent, and/or an anti-inflammatory agent. Suitable analgesic agents include, but are not limited to, a therapeutically effective amount of one or more ophthalmic analgesics, such as lidocaine, proparacaine, tetracaine, and the like present in an amount between about 0.1% and about 5% (w/v).

Suitable antipruritic agents include, but are not limited to, ophthalmic antihistamines and/or decongestants such as ketotifen, nedocromil, naphazoline/pheniramine, phenylephrine, epinastine, olopatadine, azelastine, bepotastine, alcaftadine, pemirolast, tetrahydrozoline, lodoxamide, naphazoline, tetrahydrozoline/zinc sulfate, cromolyn, emedastine, oxymetazoline, and the like.

Suitable anti-inflammatory agents include, but are not limited to, ophthalmic anti-inflammatory agents such as bromfenac, ketorolac, nepafenac, lifitegrast, cyclosporine, flurbiprofen, suprofen, diclofenac, and the like.

Each of the analgesic agent, antipruritic agent, and/or anti-inflammatory agent can be present in the composition in an amount of about 0.001% to about 5%, about 0.001% to about 4%, 0.001% to about 3%, about 0.001% to about 2%, about 0.001% to about 1%, about 0.001% to about 0.5%, about 0.001% to about 0.25%, about 0.001% to about 0.1%, about 0.001% to about 0.05%, or about 0.001% to about 0.01% (w/v) of the analgesic agent, antipruritic agent, and/or anti-inflammatory agent. In some embodiments, the composition contains about 0.0001%, 0.0005%, 0.001%, 0.005%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, or about 5% (w/v) of the analgesic agent, antipruritic agent, and/or anti-inflammatory agent.

Alternatively or additionally, topical compositions may contain a therapeutically effective amount of one or more miotics, such as carbachol or acetylcholine present in the composition in an amount between about 0.01% and about 4% (w/v). Alternatively or additionally, topical compositions may include a therapeutically effective amount of one or more ocular lubricants, such as coconut oil, carboxymethylcellulose, dextran, glycerin, hypromellose, polyethylene glycol 400 (PEG 400), polysorbate, polyvinyl alcohol, povidone, or propylene glycol.

Topical compositions may employ, as an alternative to resveratrol, or in addition to resveratrol, one or more of the following AMPK activators: metformin, AICAR, berberine, EGCG, carnitine, R-Lipoic acid, quercetin, glucosamine, curcumin, anthocyanins, cannabinoids, genistein, astragalus, reishi, rooibos, creatine, gynostemma, apigenin, hydroxytyrosol, baicalin. These additional or alternative AMPK activators can be present in an amount of 0.001% to about 5%, about 0.001% to about 4%, 0.001% to about 3%, about 0.001% to about 2%, about 0.001% to about 1%, about 0.001% to about 0.5%, about 0.001% to about 0.25%, about 0.001% to about 0.1%, about 0.001% to about 0.05%, or about 0.001% to about 0.01% (w/v) of the additional or alternative AMPK activator. In some embodiments, the composition contains about 0.0001%, 0.0005%, 0.001%, 0.005%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, or about 5% (w/v) of the additional or alternative AMPK activator.

In various embodiments, the composition can include an agent selected from the group consisting of methyl salicylate, trolamine salicylate, lidocaine, benzocaine, dibucaine, prilocaine, diclofenac sodium gel, hydrocortisone, clobetasol, diphenhydramine, and ketoprofen in an amount of 0.001% to about 5%, about 0.001% to about 4%, 0.001% to about 3%, about 0.001% to about 2%, about 0.001% to about 1%, about 0.001% to about 0.5%, about 0.001% to about 0.25%, about 0.001% to about 0.1%, about 0.001% to about 0.05%, or about 0.001% to about 0.01% (w/v) of the agent. In some embodiments, the composition contains about 0.0001%, 0.0005%, 0.001%, 0.005%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, or about 5% (w/v) of the agent.

In some embodiments, the composition includes at least one absorption enhancer. The absorption enhancer can be, without limitation, dimethyl sulfoxide (DMSO) or pluronic lecithin organogel. The absorption enhancer can be present in an amount of at least, greater than, or less than about 0.0001%, 0.0005%, 0.001%, 0.005%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 10%, 15%, or 20% (w/v) or (w/w).

### Methods of Treatment

A preclinical (i.e. animal) model of PRK exists that uses heptanol to remove the corneal epithelium and produce an abrasion that shears terminal nerve endings in the epithelium and evokes hyperalgesia, photophobia, and delayed dry eye symptoms. This preclinical model mimics the epithelial/neuronal damage produced by PRK in humans and causes key post-operative symptoms, including acute pain and chronic dry eye, making it an ideal model to test novel therapeutics to attenuate PRK pain.

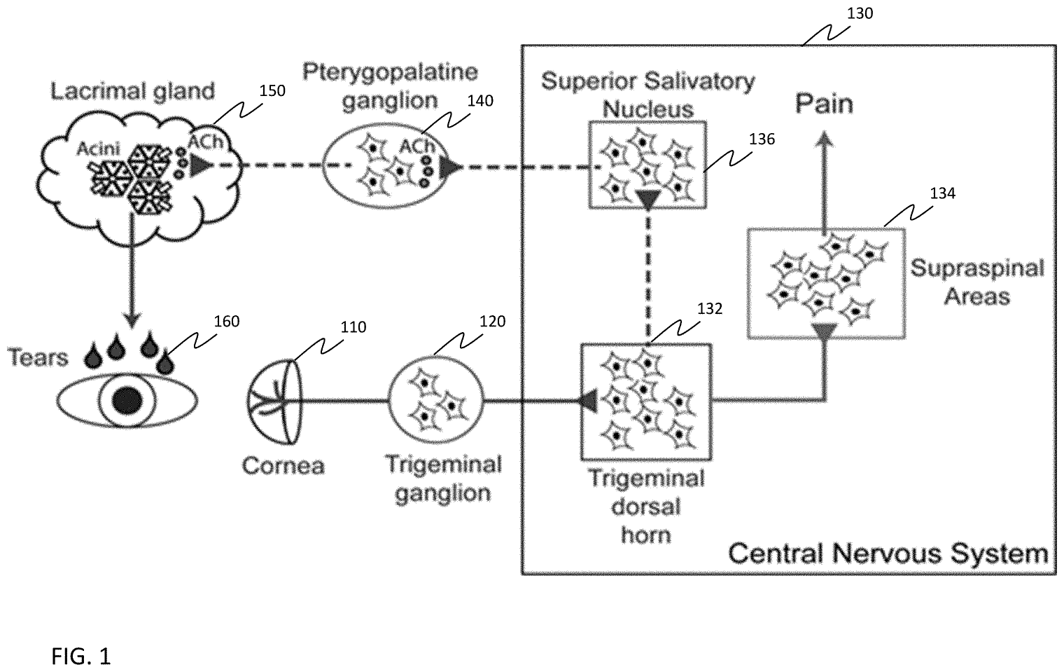

FIG. 1 is a diagram of corneal sensory nerves and reflex pathways. Sensory neurons located in the cornea 110 of an eye transmit signals through the trigeminal ganglion 120 into the central nervous system (CNS) 130. The trigeminal dorsal horn 132 conveys the signals to supraspinal areas 134, where a sensation of pain is generated. The trigeminal dorsal horn 132 also conveys the signals to the superior salivatory nucleus 136, which generates responsive signals out of the CNS and through the pterygopalatine ganglion 140 into the lacrimal gland 150, which produces tears 160 that wet the eye as shown.

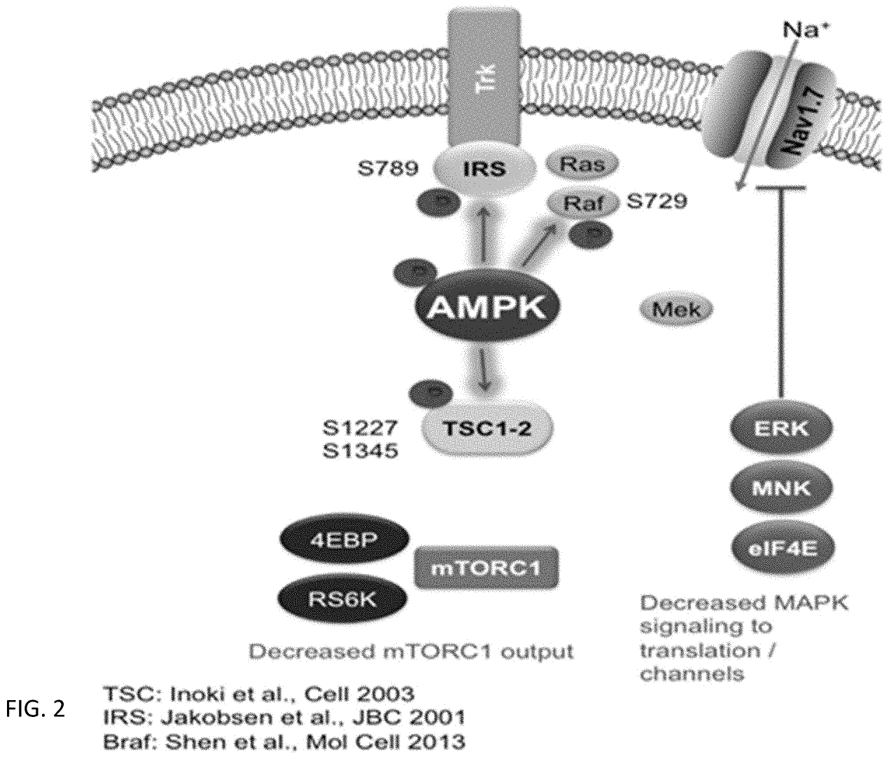

Injury to peripheral nerves activates a number of signaling pathways that contribute to pathological changes that alter the phenotype and excitability of primary sensory neurons. These signaling pathways include the mechanistic target of rapamycin (mTOR) 210 and the mitogen activated protein kinase pathways (MAPK). AMPK activation leads to suppression of both mTOR and MAPK signaling. FIG. 2, which is a diagram of AMPK signaling, illustrates the relationships among AMPK and ERK, mTOR and MAPK signaling.

In rat and mouse models of injury-induced neuropathic pain, AMPK activators fully reverse established neuropathic mechanical and cold hypersensitivity. A remarkable feature of AMPK activators is that they have disease-modifying properties, causing a complete reversal of signs of neuropathic pain for up to two months following cessation of treatment. These findings have also been reported in chemotherapy neuropathy, chronic inflammation, and diabetic neuropathy models. Hence, AMPK is an important new neuropathic pain target with disease-modifying potential.

The pain from PRK, LASIK or other ocular surgeries is likely due to severing the corneal nerve fibers and the subsequent sensitization mechanisms in the severed nerve endings. Preclinical studies support the role of sodium channels in corneal hyperalgesia since topical application of TTX can reduce the photophobia seen after heptanol abrasions of the corneal surface. However, it is unclear whether TTX can be developed as a corneal pain therapeutic as it is not soluble in tear film and has potential for toxicity. The use of other sodium channels blockers (e.g., local anesthetics) is not sufficient to reduce pain for a sustained period of time, and their repeated use causes damage to the corneal epithelium. Resveratrol is an analgesic that works through a distinct mechanism to reduce neuronal responses to acute injury (the severing of axons during corneal abrasion). Resveratrol application to cultured trigeminal ganglion neurons has a direct effect on these neurons to reduce hyperexcitability caused by nerve growth factor exposure. Without being bound by theory, it is believed that and rather than trying to reverse mechanisms that are wound up after nerve injury, treatment with the compositions described herein can prevent them from even getting started, or ameliorate or attenuate the effects of these pain-inducing mechanisms. A corneal pain therapeutic with a mechanism distinct from current therapies that attenuates post-surgical eye pain and prevents the development of chronic pain, photophobia, and dry eye represents a first-in-class therapeutic offering enormous benefit for the medical field.

Removal of the corneal epithelium with alcohol solution is used in clinical (human) procedures, such as vision correction surgery, to allow access to the underlying tissues for laser correction. The resulting corneal abrasion causes a severe acute pain due to damage to corneal nerve endings that occurs during the preparation for laser correction. Corneal abrasion pain is treated with local and systemic opioids, but is still reported as intolerable by some patients even after analgesic treatment. In preclinical procedures, the corneal epithelium is abraded with heptanol to determine if local resveratrol can attenuate the acute pain produced by this injury.

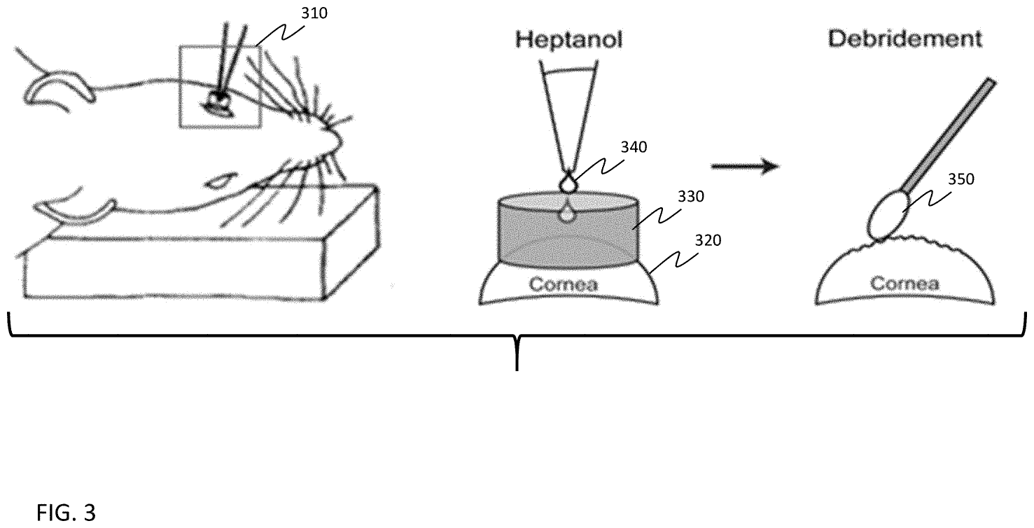

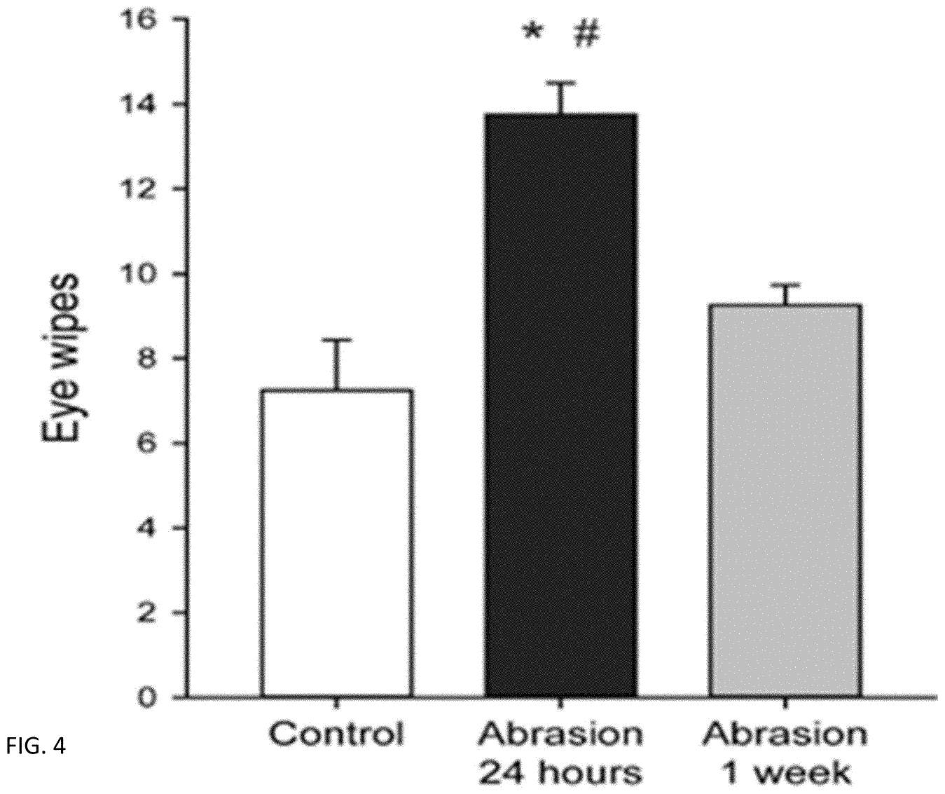

A unilateral corneal injury was performed using topical application of heptanol. FIG. 3 is a diagram of a corneal abrasion method using topical application 310 of heptanol performed on an anesthetized rat. A local anesthetic was applied to the eye 320 via ophthalmic drops. A small metal ring 330 was then secured to the corneal surface with petroleum jelly. Heptanol (10 μL) 340 was placed inside the ring for 90 seconds, the heptanol was removed from inside the ring with a cotton swab and the eye is rinsed profusely with saline. The loose corneal epithelium was then removed with a cotton applicator 350. FIG. 4 is a graph representing of acute hyperalgesia after corneal abrasion injury, illustrating that this method induces photophobia as well as hyperalgesia to ocular application of menthol. This method produces abrasions of relatively uniform size and depth, in contrast to other mechanical abrasion methods. The heptanol abrasion method causes debridement wounds, but does not remove the basement membrane.

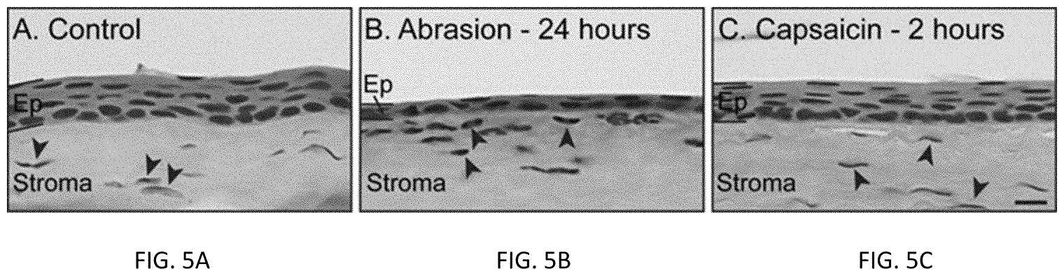

FIGS. 5A-5C are diagrams of hematoxylin and eosin (H&E) stains showing loss of epithelial cells 24 hours after abrasion injury (FIG. 5B) compared to control (FIG. 5A), illustrating that capsaicin application (FIG. 5C) does not alter overall epithelial structure. Deeper wounds may remove the basement membrane and prevent reinnervation (at least in some strains of mice). Consistent with prior photophobia studies, rats demonstrated hyperalgesia to ocular application of menthol 24 hours after corneal injury, as FIG. 4 illustrates. Using a wheel-running assay, it was confirmed that spontaneous behavior is also inhibited for 24-48 hours.

In various embodiments, topical (ophthalmic) application of the compositions described herein can reduce or eliminate the hyperalgesia produced by corneal abrasion in a pre-clinical model of PRK. Each rat received a unilateral corneal abrasion and topical treatment with eye drops containing resveratrol according to various embodiments, followed by hyperalgesic testing 24 hours and one week after surgery. Three doses of the drug were tested (see below), and experiments were conducted in both male and female rats. Control animals received vehicle eye drops to measure any placebo effects of fluid application to the eyes before surgery. Other control studies tested the effects of resveratrol alone on the corneal nociceptive responses to noxious ocular stimulation (see Table 1, below).

TABLE 1
Number of animals tested at each drug dose, including vehicle
controls, naïve controls, and both sexes.

| Drug Dose | Vehicle | 0.5% | 1% | 2% |
|---|---|---|---|---|
| PRK treated - male | 10 | 10 | 10 | 10 |
| PRK treated - female | 10 | 10 | 10 | 10 |
| Naïve controls - male | — | — | — | 10 |
| Naïve controls - female | | | | 10 |
| Subtotal | 20 | 20 | 20 | 40 |

Prior studies have shown that hyperalgesia is robust at 24 hours after injury, as FIG. 4 illustrates, and that initial stages of epithelial closure are completed in 24 hours, as FIG. 5 illustrates, making this is a feasible time for testing nociceptive responses to ocular stimulation.

Doses of trans-resveratrol up to 4% (corresponding to up to 800 μg in 10 μL) were tested. Trans-resveratrol is the more biologically active isomer of resveratrol. Topical application is efficacious, because resveratrol is very lipophilic and should be readily taken up into the tear film that covers the surface of the eye. Treatments were applied 24 hours before abrasion; one hour before abrasion; and ten minutes post abrasion. These time points can be feasible for humans planning to undergo ocular surgery. Some rats (vehicle control group) received eye drops that contain the vehicle formulation, but not resveratrol, at similar time points before abrasion surgery (see Table 1 above). These vehicle control rats underwent abrasion surgery and are tested with nociceptive ocular stimulation at 24 hours after abrasion. This group controlled for potential effects of applying eye drops to reduce nociceptive ocular responses. Both sexes were tested in each of the protocols. A separate control group (naïve) received resveratrol eye drops at similar time points but did not undergo the abrasion procedure. This group received only the highest dose of resveratrol and is used to assess the extent to which the drug suppresses nociceptive responses to ocular stimulation in uninjured controls.

It is advisable to conduct dose-response studies in both male and female rats to ensure that the effects of ocular injury and resveratrol are confirmed in both sexes, as Table 1, above, shows. Nociceptive responses (eye wipes) were assessed in response to ocular application of menthol (50 mM) at 24 hours and one week after corneal abrasion to assess hyperalgesia.

Though a variety of noxious stimuli (capsaicin, menthol, hypertonic saline) are known to evoke stereotypical eye-wipe nocifensive responses following ocular stimulation, testing was first be conducted with menthol, which has been shown to evoke hyperalgesia after abrasion, as FIG. 4 illustrates.

Testing responses to noxious stimulation of the corneal surface requires direct handling of rats, and it is best if the rats are well adapted to experimenter handling. Thus, rats are handled by the experimenter daily for three days before the first eye drop treatment to acclimate them to the handling procedure and the nociceptive testing environment. Rats are held in the cloth restraint that is used for application of noxious stimuli, and then placed in the transparent chamber, where eye wipe behaviors are assessed for five minutes each day of habituation.

Table 2, below, presents a timeline for testing. Day 0 is the time of corneal abrasion; in the afternoon after morning application of second dose of resveratrol (or vehicle) eye drops.

TABLE 2
Timeline for experimental schedule, treatment and assessments.

| Experiment Day | −3 | −2 | −1 | 0 | 1 | 7 | 8 |
|---|---|---|---|---|---|---|---|
| Handling/habituation | ✓ | ✓ | ✓ | | | | |
| Measure tear volume | | ✓ | | | | | ✓ |
| Apply resveratrol/vehicle eye drops | | | ✓ | ✓ | | | |
| Corneal abrasion injury (heptanol) | | | | ✓ | | | |
| Nociceptive testing (ocular | | | | | ✓ | ✓ | |
| application of noxious stimulus) | | | | | | | |
| Histology (following | | | | | | | ✓ |
| euthanasia and perfusion) | | | | | | | |

Per Table 2, rats are handled on the day before corneal abrasion and then receive application of resveratrol or vehicle eye drops into both eyes using the same restraint procedure that is used for testing noxious stimulation after corneal abrasion. The first treatment occurs 24 hours before corneal abrasion. Eye drop application is repeated in rats on the day of corneal abrasion (one hour before abrasion, and again ten minutes after abrasion, under anesthesia). The experimenter applying the eye drops should not be aware the treatment being applied (resveratrol or vehicle).

Twenty-four hours after corneal abrasion surgery, rats received 10 μL of liquid applied to the eye containing 50 mM menthol to test nociceptive responses. Each rat is lightly restrained in a cloth towel during application of the noxious ocular stimulation and then placed in a transparent box for assessment of eye wipe nociceptive responses. Two observers both observe and count the eye wipe behaviors together. Testing may be video recorded to allowed later review if necessary.

Dry eye disease is a complex disorder, but can include symptoms of ocular pain as well as deficient tear production. Photophobia and dry eye disease symptoms are common after vision correction surgery and can become chronic after surgery. In the pre-clinical PRK model, tear volume is reduced by 30% at one week after corneal abrasion. Since photophobia and dry eye symptoms may be a long-term consequence of corneal nerve injury, the ability of resveratrol to reduce this effect was assessed. In various embodiments, resveratrol also increases tear production; this effect alone can be of therapeutic value for dry eye—even without pain. Thus, while reducing ocular pain is advantageous, a resveratrol-based drug may also improve tear production which can indirectly reduce pain or discomfort.

Tear production may be assessed using a “Zone-Quick” phenol thread test commercially available from Oasis Medical, Inc., of Glendora, Calif. This test is equivalent to the well-known Schirmer's test. A timed wicking (15 seconds/eye) is used to measure tear production in rats briefly anesthetized with isoflurane (5% in oxygen). At the end of 15 seconds, the length of thread that turned red is measured to the nearest millimeter (mm). Each animal is tested before abrasion (baseline) and at one-week post-abrasion. The phenol thread test is done at least 24 hours before nociceptive testing, because even brief anesthesia interferes with nociceptive testing on the same day.

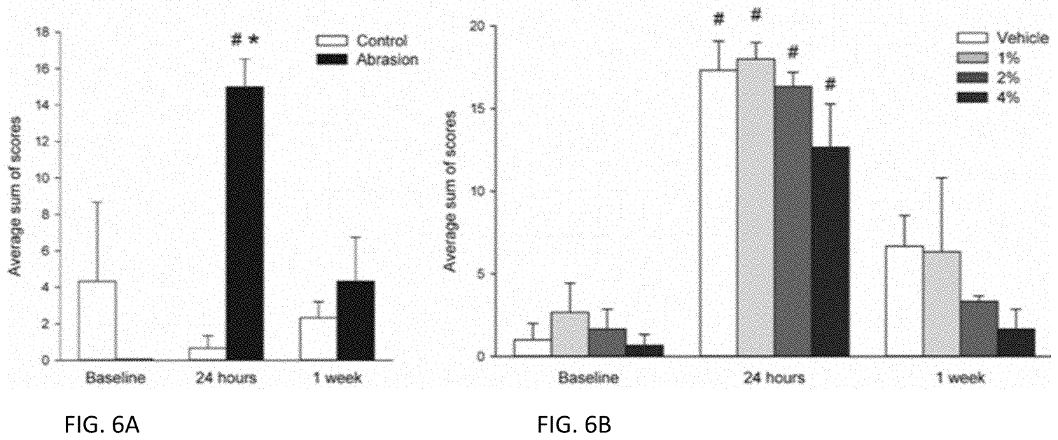

FIGS. 6A and 6B are graphs showing efficacy of resveratrol medication for the treatment of ocular pain. The y-axes of FIGS. 6A and 6B are orbital tightening (OT) after the corneal abrasion injury that is designed to mimic PRK surgery. As those skilled in the art understand, OT is a behavioral indicator of acute ocular pain, chronic ocular pain, dry eye disease, or photophobia. For more information regarding OT and illustrations of animals exhibiting OT, see, Chambers, et al., “Ontogeny and Phylogeny of Facial Expression of Pain,” Pain 156 (5): 798-799 (2015). The abrasion increases OT scores at 24 hours post-injury. FIG. 6B shows a dose-dependent trend for resveratrol to reverse this. Larger sample sizes are expected to show more a pronounced trend.

It is also apparent that ocular pain, dryness, photophobia, or any combination thereof, may be relieved by a method in which one or more eye drops containing resveratrol in some concentration are made to come into contact with at least one eye. In some embodiments, the medication and method employ trans-resveratrol to treat ocular pain. In some embodiments, the method is repeated as needed or desired to effect relief, and perhaps substantial permanent resolution, of the pain, and perhaps the photophobia and dryness as well.

It is also apparent that ocular pain, dryness, photophobia, or any combination thereof, may be relieved by instilling a contact lens infused with resveratrol on at least one eye, whereby the contact lens remains on the at least one eye over time, allowing the resveratrol to come into contact with the at least one eye. Thus the disclosure also contemplates a method of infusing resveratrol, and other liquid or gel active ingredients and/or excipients, into a silicone hydrogel contact lens to render the contact lens suitable for use in controlling ocular pain, photophobia and/or dryness. In various embodiments, a silicone hydrogel contact lens includes any of the amounts of resveratrol described herein either (w/w) or (w/v).

The disclosure includes a method of treating an eye disorder chosen from acute ocular pain, chronic ocular pain, dry eye disease, and/or photophobia using the topical compositions described herein. The methods described herein include administering to the subject a therapeutically effective amount of at least one compound described herein formulated in a topical pharmaceutical composition. In various embodiments, a therapeutically effective amount of at least one compound described herein present in a pharmaceutical composition is the only therapeutically active compound in a pharmaceutical composition. In certain embodiments, the method further comprises administering to the subject an additional therapeutic agent that treats inflammation, acute ocular pain, chronic ocular pain, dry eye disease, and/or photophobia. In some embodiments, pretreatment and perioperative treatment of the ocular surface with the compositions described herein reduces corneal abrasion hyperalgesia.

In certain embodiments, administering the compound(s) described herein to the subject allows for administering a lower dose of the additional therapeutic agent as compared to the dose of the additional therapeutic agent alone that is required to achieve similar results in treating acute ocular pain, chronic ocular pain, dry eye disease, and/or photophobia in the subject. For example, in certain embodiments, the compound(s) described herein enhance(s) the activity of the additional therapeutic compound, thereby allowing for a lower dose of the additional therapeutic compound to provide the same effect. In certain embodiments, the compound(s) described herein, and the therapeutic agent are co-administered to the subject. In other embodiments, the compound(s) described herein, and the therapeutic agent are coformulated and co-administered to the subject.

In some embodiments, the additional therapeutic agent includes an ophthalmic (topical) NSAID (non-steroidal anti-inflammatory drug), which can block the cyclooxygenase pathway and inhibit the formation of inflammatory mediators causing pain. In certain embodiments, the NSAID is selected from the group consisting of diclofenac, ketorolac, bromfenac, or nepafenac, and may provide synergistic efficacy in treating ocular pain. In another embodiment, the additional therapeutic agent includes an ophthalmic (topical) corticosteroid. The corticosteroid, in some embodiments, is selected from the group consisting of loteprednol etabonate, fluocinolone, fluorometholone, dexamethasone, prednisolone, difluprednate, and rimexolone.

The use of a corticosteroid in combination with resveratrol can provide synergistic efficacy in treating ocular pain. In another embodiment, a synergistic combination of resveratrol with the therapeutic use of a silicone hydrogel contact lenses or bandage contact lenses may improve efficacy of relieving ocular pain by cushioning exposed corneal nerves to protect the cornea from mechanical irritation and facilitate wound healing. In some embodiments, resveratrol and a single NSAID are the only two therapeutically active agents in the composition. In some embodiments, resveratrol and a single corticosteroid are the only two therapeutically active agents in the composition. In certain embodiments, the subject is a mammal. In other embodiments, the mammal is a human.

### Administration/Dosage/Formulations

The regimen of administration may affect what constitutes an effective amount. The therapeutic formulations may be administered to the subject either prior to or after the onset of acute ocular pain, chronic ocular pain, dry eye disease, and/or photophobia. Further, several divided dosages, as well as staggered dosages may be administered daily or sequentially. Further, the dosages of the therapeutic formulations may be proportionally increased or decreased as indicated by the exigencies of the therapeutic or prophylactic situation.

Administration of the compositions described herein to a patient, preferably a mammal, more preferably a human, may be carried out using known procedures, at dosages and for periods of time effective to treat acute ocular pain, chronic ocular pain, dry eye disease, and/or photophobia in the patient. An effective amount of the therapeutic compound necessary to achieve a therapeutic effect may vary according to factors such as the state of the disease or disorder in the patient; the age, sex, and weight of the patient; and the ability of the therapeutic compound to treat acute ocular pain, chronic ocular pain, dry eye disease, and/or photophobia in the patient. Dosage regimens may be adjusted to provide the optimum therapeutic response. For example, several divided doses may be administered daily, or the dose may be proportionally reduced as indicated by the exigencies of the therapeutic situation. A non-limiting example of an effective dose range for a therapeutic compound described herein is from about 1 to about 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well known in the medical arts.

A medical doctor, e.g., physician or veterinarian, having ordinary skill in the art may readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the compound(s) described herein are dictated by and directly dependent on (a) the unique characteristics of the therapeutic compound and the particular therapeutic effect to be achieved, and (b) the limitations inherent in the art of compounding/formulating such a therapeutic compound.

In certain embodiments, the compositions described herein are administered to the patient in dosages that range from one to five times per day or more. In other embodiments, the compositions described herein are administered to the patient in range of dosages that include, but are not limited to, once every day, every two days, every three days, once a week, and once every two weeks. It is readily apparent to one skilled in the art that the frequency of administration of the various combination compositions described herein varies from individual to individual depending on many factors including, but not limited to, age, disease or disorder to be treated, gender, overall health, and other factors. Thus, administration of the compounds and compositions described herein should not be construed to be limited to any particular dosage regime and the precise dosage and composition to be administered to any patient is determined by the attending physician taking all other factors about the patient into account.

The compound(s) described herein for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound described herein is from about 1 mg to about 2,500 mg. In some embodiments, a dose of a compound described herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, a composition as described herein is a packaged pharmaceutical composition comprising a container holding a therapeutically effective amount of a compound described herein, alone or in combination with a second pharmaceutical agent; and instructions for using the compound to treat, prevent, or reduce one or more symptoms of acute ocular pain, chronic ocular pain, dry eye disease, and/or photophobia in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

### Dosing

The therapeutically effective amount or dose of a compound described herein depends on the age, sex and weight of the patient, the current medical condition of the patient and the progression of acute ocular pain, chronic ocular pain, dry eye disease, and/or photophobia in the patient being treated. The skilled artisan is able to determine appropriate dosages depending on these and other factors.

A suitable dose of a compound described herein can be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the compound(s) described herein is optionally given continuously; alternatively, the dose of drug being administered is temporarily reduced or temporarily suspended for a certain length of time (i.e. a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease state is retained. In certain embodiments, patients require intermittent treatment on a long-term basis upon any recurrence of symptoms and/or infection.

The compounds described herein can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

### Enumerated Embodiments

The following exemplary embodiments are provided, the numbering of which is not to be construed as designating levels of importance:

Embodiment 1 provides an ophthalmic composition comprising:

about 0.001% to about 5% (w/v) resveratrol;

about 0.001% to about 5% (w/v) of at least one pharmaceutically acceptable excipient;

and an aqueous vehicle.

Embodiment 2 provides the composition of embodiment 1, wherein the resveratrol is at least 95% trans-resveratrol.

Embodiment 3 provides the composition of any one of embodiments 1-2, wherein the at least one pharmaceutically acceptable excipient is selected from the group consisting of polyvinylpyrrolidone (PVP), polyvinyl alcohol, polyvinyl acrylic acid, hydroxypropylmethyl cellulose, sodium carboxymethyl cellulose, and xanthan gum, a preservative, a chelating agent, a buffering agent, a surfactant, and an antioxidant.

Embodiment 4 provides the composition of any one of embodiments 1-3, which has an osmolality of about 200 to about 400 mOsm.

Embodiment 5 provides the composition of any one of embodiments 1-4, which has a viscosity of about 0.5 to about 5 cP.

Embodiment 6 provides the composition of any one of embodiments 1-5, which has a pH of about 6.5 to about 7.5.

Embodiment 7 provides the composition of any one of embodiments 1-6, wherein the aqueous vehicle is water.

Embodiment 8 provides the composition of any one of embodiments 1-7, further comprising at least one of:

an analgesic agent, an antipruritic agent, and an anti-inflammatory agent.

Embodiment 9 provides the composition of any one of embodiments 1-8, further comprising an agent selected from the group consisting of methyl salicylate, trolamine salicylate, lidocaine, benzocaine, dibucaine, prilocaine, diclofenac sodium gel, hydrocortisone, clobetasol, diphenhydramine, and ketoprofen.

Embodiment 10 provides the composition of any one of embodiments 1-9, further comprising an AMPK activator selected from the group consisting of metformin, AICAR, berberine, EGCG, carnitine, R-Lipoic acid, quercetin, glucosamine, curcumin, anthocyanins, cannabinoids, genistein, astragalus, reishi, rooibos, creatine, gynostemma, apigenin, hydroxytyrosol, and baicalin.

Embodiment 11 provides the composition of any one of embodiments 1-10, further comprising at least one of dimethyl sulfoxide and a lecithin organogel.

Embodiment 12 provides the composition of any one of embodiments 1-11, which is formulated as an eye drop or ointment.

Embodiment 13 provides method of treating acute ocular pain, chronic ocular pain, dry eye disease, or photophobia, the method comprising administering the composition of claim 1 to an eye of a subject in need thereof.

Embodiment 14 provides the method of embodiment 13, wherein the composition is an eye drop or ointment.

Embodiment 15 provides the method of any one of embodiments 13-14, wherein the resveratrol is the only therapeutically active agent in the composition.

Embodiment 16 provides the method of any one of embodiments 13-15, wherein the composition comprises an additional therapeutic agent.

Embodiment 17 provides the method of any one of embodiments 13-16, wherein the additional therapeutic agent is an NSAID or a corticosteroid.

Embodiment 18 provides the method of any one of embodiments 13-17, wherein the NSAID is selected from the group consisting of diclofenac, ketorolac, bromfenac, and nepafenac.

Embodiment 19 provides the method of any one of embodiments 13-18, wherein the corticosteroid is selected form the group consisting of loteprednol etabonate, fluocinolone, fluorometholone, dexamethasone, prednisolone, difluprednate, and rimexolone.

Embodiment 20 provides the method of any one of embodiments 13-19, wherein the resveratrol and the NSAID are the only two therapeutically active agents in the composition.

Embodiment 21 provides the method of any one of embodiments 13-20, wherein the resveratrol and the corticosteroid are the only two therapeutically active agents in the composition.

What is claimed is:

1. An ophthalmic composition comprising:
about 0.001% to about 5% (w/v) resveratrol;
about 0.001% to about 5% (w/v) of at least one pharmaceutically acceptable excipient; and
an aqueous vehicle.

2. The composition of claim 1, wherein the resveratrol is at least 95% trans-resveratrol.

3. The composition of claim 1, wherein the at least one pharmaceutically acceptable excipient is selected from the group consisting of polyvinylpyrrolidone (PVP), polyvinyl alcohol, polyvinyl acrylic acid, hydroxypropylmethyl cellulose, sodium carboxymethyl cellulose, and xanthan gum, a preservative, a chelating agent, a buffering agent, a surfactant, and an antioxidant.

4. The composition of claim 1, which has an osmolality of about 200 to about 400 mOsm.

5. The composition of claim 1, which has a viscosity of about 0.5 to about 5 cP.

6. The composition of claim 1, which has a pH of about 6.5 to about 7.5.

7. The composition of claim 1, wherein the aqueous vehicle is water.

8. The composition of claim 1, further comprising at least one of:
an analgesic agent, an antipruritic agent, and an anti-inflammatory agent.

9. The composition of claim 1, further comprising an agent selected from the group consisting of methyl salicylate, trolamine salicylate, lidocaine, benzocaine, dibucaine, prilocaine, diclofenac sodium gel, hydrocortisone, clobetasol, diphenhydramine, and ketoprofen.

10. The composition of claim 1, further comprising an AMPK activator selected from the group consisting of metformin, AICAR, berberine, EGCG, carnitine, R-Lipoic acid, quercetin, glucosamine, curcumin, anthocyanins, cannabinoids, genistein, astragalus, reishi, rooibos, creatine, gynostemma, apigenin, hydroxytyrosol, and baicalin.

11. The composition of claim 1, further comprising at least one of dimethyl sulfoxide and a lecithin organogel.

12. The composition of claim 1, which is formulated as an eye drop or ointment.

13. A method of treating acute ocular pain, chronic ocular pain, dry eye disease, or photophobia, the method comprising administering the composition of claim 1 to an eye of a subject in need thereof.

14. The method of claim 13, wherein the composition is an eye drop or ointment.

15. The method of claim 13, wherein the resveratrol is the only therapeutically active agent in the composition.

16. The method of claim 13, wherein the composition comprises an additional therapeutic agent.

17. The method of claim 16, wherein the additional therapeutic agent is an NSAID or a corticosteroid.

18. The method of claim 17, wherein the NSAID is selected from the group consisting of diclofenac, ketorolac, bromfenac, and nepafenac.

19. The method of claim 17, wherein the corticosteroid is selected form the group consisting of loteprednol etabonate, fluocinolone, fluorometholone, dexamethasone, prednisolone, difluprednate, and rimexolone.

20. The method of claim 17, wherein the resveratrol and the NSAID are the only two therapeutically active agents in the composition.

21. The method of claim 17, wherein the resveratrol and the corticosteroid are the only two therapeutically active agents in the composition.
